(19) United States
(12) Patent Application Publication
(10) Pub. No.: US 2024/0158739 A1
(43) Pub. Date: May 16, 2024

(54) ADHESION IMPROVER CONTAINING AN EDIBLE PLANT-DERIVED COMPONENT

(71) Applicant: NITTO DENKO CORPORATION, Ibaraki-shi, Osaka (JP)

(72) Inventors: Yuka SEKIGUCHI, Ibaraki-shi, Osaka (JP); Tomoko SUDO, Ibaraki-shi, Osaka (JP); Tomoaki HISHIKI, Ibaraki-shi, Osaka (JP); Takahisa KONISHI, Ibaraki-shi, Osaka (JP); Shinya HASHIGUCHI, Ibaraki-shi, Osaka (JP)

(21) Appl. No.: 18/284,651

(22) PCT Filed: Mar. 31, 2022
(86) PCT No.: PCT/JP2022/016588

§ 371 Date: Sep. 28, 2023

(30) Foreign Application Priority Data
Mar. 31, 2021 (JP) 2021-061250

(51) Int. Cl. C12N 5/00; A23J 3/16

(52) U.S. Cl. CPC C12N 5/0068; A23J 3/16; C12N 2533/50; C12N 2533/74; C12N 2539/00

(57) ABSTRACT

The purpose of the present invention is to provide: an adhesion improver which contains an edible plant-derived component and is used in an edible base material for culturing cells; a cell culturing scaffolding material containing said adhesion improver; and a tissue body containing said cell culturing scaffolding material.

A cell culturing scaffolding material suitable for producing cultured meat can be produced by applying an adhesion improver containing an edible plant-derived component to an edible base material for culturing cells.

### TECHNICAL FIELD

The present invention relates to: an adhesion improver which contains an edible plant-derived component and is used in an edible base material for culturing cells; a cell culturing scaffolding material containing said adhesion improver; and a tissue body containing said cell culturing scaffolding material.

### BACKGROUND ART

In recent years, the demand for meat is expected to increase as the world population grows. In order to meet future demand for meat, it is not enough to simply increase manufacturing efficiency to increase manufacturing volume of conventional protein sources and it is indispensable to develop new protein sources. The new protein sources include plant-based meat manufactured from plants, meat manufactured from insects, and cultured meat manufactured by culturing microorganisms or cells themselves.

“Cultured meat” refers to a meat produced by culturing muscle cells using regenerative medicine technology, which is also called as “cultured meat” or “clean meat. One of the advantages of the cultured meat is safety. For example, in a process of manufacturing and processing meat, there is always some risks of contamination of pathogenic bacteria that may cause food poisoning. However, the cultured meat is cultured under nearly sterile condition, so the risk of contamination of pathogenic bacteria is lower. Using the cultured meat can reduce greenhouse gas emissions by 96% compared to conventional producing methods, in addition to reducing the cost required for processing process according to a result of study, which attracts attention from the environmental perspective.

During producing of cultured meat, it is desirable not to use animal-derived materials such as gelatin and collagen for its purpose. Those using soybean defatted tofu cheese (refer to Patent Document 1) and textured soy protein (i.e., soy meat, refer to Patent Document 2) have been known as scaffolding material for cell culturing using plant-derived materials.

However, although soybean defatted tofu cheese is porous, the pore size is too small (around 200 nm) for a cell to enter into. Therefore, it is only on the surface of the soybean defatted tofu cheese where cells adhere and are cultured, and soybean defatted tofu cheese cannot be used to culture cells three-dimensionally. In addition, the soybean defatted tofu cheese is not good at cell adhesion.

While a scaffolding material made from soy meat is porous and possible to be used for three-dimensional culture, almost of products using the scaffolding material are in flake or granule form, and it is difficult to control it to have the desired shape and texture such as steak meat, sashimi, or fillet. In addition, also the soy meat is not good at cell adhesion.

### PRIOR ART REFERENCES

### Patent References

Patent Document 1: Japanese Unexamined Patent Application Publication (Translation of PCT Application) No. 2004-518771
Patent Document 2: Japanese Unexamined Patent Application Publication (Translation of PCT Application) No. 2020-527054

### SUMMARY OF INVENTION

### Problems to be Solved by Invention

A purpose of the present invention is to provide: an adhesion improver which contains an edible plant-derived component and is used in an edible base material for culturing cells; a cell culturing scaffolding material containing said adhesion improver; and a tissue body containing said cell culturing scaffolding material.

### MEANS OF SOLVING PROBLEMS

It is required to culture muscle cells three-dimensionally using a scaffold material in order to produce cultured meat controlled to have a desired shape and texture, such as steak meat, sashimi, or fillet. Also, during producing of cultured meat, it is desirable not to use animal-derived materials such as gelatin and collagen for its purpose.

Although some scaffolding materials not using animal-derived materials have been known so far, there have been no scaffolding materials that are capable of three-dimensional culture, easy to control shape and texture, and good at cell adhesion.

The inventors have found, as a result of diligent researches to solve the aforementioned problems, that a cell culturing scaffolding material suitable for producing cultured meat can be produced by applying an adhesion improver containing an edible plant-derived component to an edible base material for culturing cells, and further studies have been conducted to complete the present invention.

In other words, the present invention relates to:
(1) An adhesion improver containing edible plant-derived component used in an edible base material for cell culturing;
(2) The adhesion improver according to (1), wherein the edible plant-derived component is seed-derived;
(3) The adhesion improver according to (2), wherein the seed is a Fabaceae plant seed;
(4) The adhesion improver according to (3), wherein the seed is a soybean; (5) The adhesion improver according to any one of (1) to (4), wherein the edible plant-derived component is isolated soy protein;
(6) The adhesion improver according to any one of (1) to (5), wherein the edible base material contains alginic acid or alginate, glucomannan, or cellulose derivative;
(7) The adhesion improver according to any one of (1) to (6), wherein the edible base material is a porous material;
(8) A cell culturing scaffolding material containing the edible base material for cell culturing and the adhesion improver according to any one of (1) to (7);
(9) The cell culturing scaffolding material according to (8), wherein a layer containing said adhesion improver is laminated on a surface of the edible base material;
(10) The cell culturing scaffolding material according to (9), wherein a thickness of the layer containing said adhesion improver is 1 nm or more.
(11) A tissue body in which cells are cultured on the cell culturing scaffolding material according to any one of (8) to (10);
(12) The tissue body according to (11), wherein the cell is derived from mammal, fish, or crustacean; and
(13) The tissue body according to (11) or (12), wherein it is for food.

### Advantageous Effects of Invention

The adhesion improver of the present invention can improve adhesion between cell culturing base materials and cells. Applying the adhesion improver of the present invention onto an edible base material in which a desired shape and texture are achieved makes it possible to produce a cell culturing scaffolding material in which the desired shape and texture are achieved, and further, which makes it possible to produce a cultured meat in which a desired shape and texture are achieved.

Furthermore, the adhesion improver of the present invention can be used in a scene where cell culturing is performed, and can be used not only in the field of cultured meat, but also in the field of pharmaceutical raw material manufacture and/or chemical raw material manufacture.

### BRIEF DESCRIPTION OF DRAWINGS

FIG. 1 is a three-dimensional image of a sample in example 1.

FIG. 2 is a three-dimensional image of a sample in example 2.

FIG. 3 is a three-dimensional image of a sample in example 3.

FIG. 4 is a three-dimensional image of a sample in example 4.

FIG. 5 is a three-dimensional image of a sample in example 5.

FIG. 6 is a three-dimensional image of a sample in example 6.

FIG. 7 is a three-dimensional image of a sample in example 7.

FIG. 8 is a three-dimensional image of a sample in example 8.

FIG. 9 is a three-dimensional image of a sample in example 9.

FIG. 10(A) is a two-dimensional image of a sample in example 10. FIG. 10(B) is a double magnified image of FIG. 10(A).

FIG. 11 is a three-dimensional image of a sample in example 11.

FIG. 12 is a three-dimensional image of a sample in reference example.

FIG. 13 is a three-dimensional image of a sample in Comparative Example 1.

FIG. 14 is a three-dimensional image of a sample in Comparative Example 2.

FIG. 15 is a three-dimensional image of a sample in Comparative Example 3.

FIG. 16 are TOF-SIMS images of specimens in Example 1.

FIG. 17 are SEM images of specimens in Example 1 and 2.

### EMBODIMENTS FOR CARRYING OUT INVENTION

The present invention is described in detail below.

Unless otherwise defined herein, all technical and scientific terms used herein have the same meanings as those ordinarily understood by those skilled in the art. All patents, applications and other publications and information referenced herein are incorporated herein by reference in their entirety. Moreover, in the event of any inconsistency between the publication referenced herein and the description herein, the description herein shall prevail.

The present invention relates to an adhesion improver containing edible plant-derived component used in an edible base material for cell culturing.

In the present invention, “edible” means that it can be safely ingested into a body, and that it consists only of substances, for example, that are identified as food or food additives by law, etc. of each country. So far as it is edible, it may or may not be absorbed into the body, and may or may not be digestible.

In the present invention, “edible base material for cell culturing” means a base material used for cell culturing, and that can be safely ingested into a body, and that the base material consists only of substances, for example, that are identified as food or food additives by law, etc. of each country.

In one embodiment of the present invention, the edible base material of the present invention is chewable and can be broken/shredded into smaller pieces by chewing before swallowing.

In the present invention, the edible base material with physical properties (e.g., Young's modulus, viscosity, robustness, etc.) corresponding to desired use (e.g., consumed by human adults) can be selected.

In the present invention, “edible plant-derived component” means a component whose raw material is plant and that is edible. In the present invention, “plant” means plant in the broadest sense including fungi and algae, but not animal. In the present invention, raw material of edible plant-derived component is not particularly limited, but is derived from, for example, seeds, roots, stems, and leaves of plants. In one embodiment of the present invention, the edible plant-derived component is a seed-derived component.

In the present invention, plants used as raw material for the edible plant-derived component are not particularly limited, but include, for example, plants belonging to Fabaceae, Vitaceae, Poaceae, Asteraceae, Arecaceae, Cottonaceae, Brassicaceae, Papaveraceae, Pedaliaceae, Rosaceae, Oleaceae, Malvaceae, Pinaceae, Polygonaceae, Ericaceae, Grossulariaceae, Zingiberaceae, Ginkgoaceae, Brassicaceae, Hippocrateaceae, Araliaceae, Theaceae, Oleaceae, Plantaginaceae, Tricholomataceae, Pleurotaceae, Trichocomaceae, Saccharomycetaceae, etc., preferably they are Fabaceae plant and Vitaceae plant, most preferably Fabaceae plant.

In the present invention, the Fabaceae plants used as raw material for the edible plant-derived component are not particularly limited, but are, for example, plants belonging to Faboideae or Papilionoideae, and include, for example, glycine max, pisum, chickpea, Phaseolus vulgaris, Vicia faba, Aarachis hypogaea, lentil, pigeon pea, mung bean, green gram, azuki bean, cowpea, cajan, etc., preferably they are soybean and garden pea, most preferably soybean and Vicia faba.

In the present invention, the Malvaceae plants used as raw material for the edible plant-derived component are not particularly limited, but include, for example, cacao, gossypium, etc.

In the present invention, the Polygonaceae plants used as raw material for the edible plant-derived component are not particularly limited, but include, for example, fagopyrum esculentum, etc.

In the present invention, the Ericaceae plants used as raw material for the edible plant-derived component are not particularly limited, but include, for example, blueberry plant and vaccinium myrtillus, etc.

In the present invention, the Grossulariaceae plants used as raw material for the edible plant-derived component are not particularly limited, but include, for example, cassis, etc.

In the present invention, the Zingiberaceae plants used as raw material for the edible plant-derived component are not particularly limited, but include, for example, ginger, curcuma, etc.

In the present invention, the Poaceae plants used as raw material for the edible plant-derived component are not particularly limited, but include, for example, Zea mays, etc.

In the present invention, the Ginkgoaceae plants used as raw material for the edible plant-derived component are not particularly limited, but include, for example, Ginkgo biloba, etc.

In the present invention, the Brassicaceae plants used as raw material for the edible plant-derived component are not particularly limited, but include, for example maca, etc.

In the present invention, the Hippocrateaceae plants used as raw material for the edible plant-derived component are not particularly limited, but include, for example, salasia, etc.

In the present invention, the Araliaceae plants used as raw material for the edible plant-derived component are not particularly limited, but include, for example American ginseng, Panax ginseng, etc. American ginseng is also called “American carrot”, “Western ginseng,” and “Cantonese ginseng”. Panax ginseng is also called Korean ginseng or Korean carrot.

In the present invention, the Theaceae plants used as raw material for the edible plant-derived component are not particularly limited, but include, for example, Camellia sinensis, etc.

In the present invention, the Oleaceae plants used as raw material for the edible plant-derived component are not particularly limited, but include, for example, olive, etc.

In the present invention, the Plantaginaceae plants used as raw material for the edible plant-derived component are not particularly limited, but include, for example, sand plantain, etc. Sand plantain is also called psyllium. Edible plant-derived component derived from psyllium includes, for example, psyllium seed gum derived from psyllium.

In the present invention, the Tricholomataceae plants used as raw material for the edible plant-derived component are not particularly limited, but include, for example, shiitake mushroom, etc.

In the present invention, the Pleurotaceae plants used as raw material for the edible plant-derived component are not particularly limited, but include, for example, pleurotus cornucopiae, etc.

In the present invention, the Trichocomaceae plants used as raw material for the edible plant-derived component are not particularly limited, but include, for example, Aspergillus oryzae, etc. Edible plant-derived component derived from Aspergillus oryzae includes, for example, tannase derived from Aspergillus oryzae.

In the present invention, the Saccharomycetaceae plants used as raw material for the edible plant-derived component are not particularly limited, but include, for example, Saccharomyces cerevisiae, etc. Saccharomyces cerevisiae is also called “ferment” or “yeast”.

In the present invention, the Vitaceae plants used as raw material for the edible plant-derived component are not particularly limited, but include, for example Vitis vinifera, fox grape, ampelopsis, etc. and it is preferably Vitis vinifera.

In the present invention, the Poaceae plants used as raw material for the edible plant-derived component are not particularly limited, but include, for example, triticum sativum, Hordeum vulgare, Zea mays, avena fatua, Oryza sativa, Secale cereale, millet, foxtail millet, echinochloa frumentacea, adlay and saccharum, etc., and it is preferably wheat.

In one embodiment of the present invention, the raw material is a Fabaceae plant, more preferably the seeds of Fabaceae plant, most preferably seeds of glycine max (i.e. soybean) or seeds of Vicia faba (i.e. broad bean).

In one embodiment of the present invention, the edible plant-derived component is a component of processed soybean. The components of processed soybean are not particularly limited, but include, for example, defatted soybean, defatted soybean milk, isolated soy protein, whey, soy meat, tofu cheese made by agglomerating defatted soy milk with calcium ions, etc., soybean saponin, soy peptone, etc., and it is preferably isolated soy protein.

Defatted soybean is soybean from which oil has been removed, and contains, for example, by weight percent, 50% or more of protein, 35% or more of carbohydrate, and 19% or less of fat.

Defatted soy milk is water-extracted fraction of defatted soybean, and the dried substance of the fraction contains, for example, by weight percent, 59.0% or more of protein, 26.9% or more of carbohydrate, and 0.2% or less of fat.

Isolated soy protein is protein isolated from defatted soy milk by isoelectric precipitation or heating, and contains, for example, by weight percent, 58.9 to 91.5% of protein, 2.2% to 31.3% of carbohydrate, and 0.2 to 31% of fat.

Whey is fraction in which isolated soy protein is removed from defatted soy milk, and contains oligosaccharide and mineral.

Soy meat is processed from the water-insoluble component of defatted soy milk and contains, for example, by weight percent, 41.4 to 74.8% of protein, 15.5 to 44.5% of carbohydrate, and 0.5 to 2.8% of fat.

Tofu cheese is made by agglutinating defatted soy milk with calcium ions, etc., and contains, for example, by weight percent, 2.1% of protein, 27.1 to 28.3% of carbohydrate, and 24.6 to 24.9% of fat.

In one embodiment of the present invention, the edible plant-derived component is a processed component in which fiber and starch are removed from seed of yellow pea plant (i.e. yellow pea) and protein is condensed, and contains, for example, by weight percent, 84% of protein, 3% of carbohydrate, 1% of fat, and 15% of ash and mineral.

In one embodiment of the present invention, the edible plant-derived component is a component processed from seed of Vitis vinifera. The component processed from seed of Vitis vinifera is not particularly limited, but for example, is extract of seed of Vitis vinifera. The extract of seed of Vitis vinifera is not particularly limited, but contains, for example, flavanols, and the flavanol contains proanthocyanidins. The extract of seed of Vitis vinifera of the present invention contains, for example, 90% of proanthocyanidin.

In one embodiment of the present invention, the edible plant-derived component is a component processed from triticum sativum seed (wheat). The component processed from wheat seed is not particularly limited, but include, for example, wheat protein, which contains, for example, gliadin and glutenin.

In the present invention, a material of the edible base material is not particularly limited, but it is preferable not to use animal-derived material. In the present invention, the edible base material contains, for example, natural polymer polysaccharides such as alginic acid or alginate, glucomannan, cellulose derivative, amylose, pectin, glucomannan, agarose, carrageenan, and locust bean gum, microorganism producing polysaccharides such as bacterial cellulose, xanthan gum, gellan, pullulan, and hyaluronic acid, and microorganism producing polyamino acids such as polyglutamic acid and polylysine, preferably contains alginic acid or alginate, glucomannan, or cellulose derivative.

In the present invention, alginate is, for example, a salt of alginic acid and a divalent metal ion. For example, in alginate, at least one G-block contained in the alginic acid forms an ionic bond with a divalent metal ion. In other words, in the alginate contained in foam, the alginic acid forms a partial salt with the divalent metal ion. The alginate, for example, have a cross-linked structure mediated by divalent metal ion. The divalent metal ion includes calcium ion, barium ion, iron ion, zinc ion, copper ion, aluminum ion, etc. and calcium ion is preferred.

In the present invention, the edible base material may further contain other polysaccharide (P) other than alginic acid and alginate, and it is preferable to contain two or more other polysaccharides (P). In the foam, the two or more other polysaccharides (P) may associate with each other. The other polysaccharide (P), for example, is that which acts as a foaming agent when the foam is made. The foam contains, as other polysaccharide (P), for example, at least one selected from the group consisting of glucomannan (konjak mannan) and cellulose derivative, preferably contains both glucomannan and cellulose derivative.

In the present invention, glucomannan is a polysaccharide contained in konjak root, etc. and has structural unit derived from glucose (glucose unit) and that derived from mannose (mannose unit). In glucomannan, each structural unit is linked mediated by 1,4-glycoside bond. In the glucomannan, the molar ratio of mannose units to glucose units is not particularly limited, but, for example, 0.5 to 2 or may be 0.5 to 1.6.

In the present invention, cellulose derivative has a structure in which a substituent has been introduced into cellulose. The substituent preferably functions as a hydrophobic group in the cellulose derivative. The cellulose derivative includes, for example, cellulose ether. The cellulose ether includes, for example, alkyl cellulose such as methyl cellulose (MC); hydroxyalkyl cellulose such as hydroxypropyl cellulose (HPC) and hydroxyethyl cellulose (HEC); hydroxyalkyl alkyl cellulose such as hydroxypropyl methyl cellulose (HPMC); carboxyalkyl cellulose such as carboxymethyl cellulose (CMC). The cellulose derivative preferably contains hydroxypropyl methyl cellulose.

In one embodiment of the present invention, the edible base material is, for example, a porous material including multiple pores. The multiple pores, for example, are continuously formed in three dimensions, and, for example, has multiple continuous pores. However, the edible base material may further have independent pore in addition to the continuous pores.

In one embodiment of the present invention, the edible base material is not particularly limited, but is, for example, a non-woven fabric, perforated sheet, foam, etc., having multiple pores.

Average pore diameter of the pores included in the edible base material is not particularly limited, but is, for example, 50 μm to 2000 μm, preferably 50 μm to 1000 μm. For the average pore diameter of the edible base material, an area of a specific pore can be calculated by image processing in an electron microscope image of a cross section of the edible base material, and a diameter of a circle having the same area as the calculated area can be considered as the pore diameter (the diameter of the pore) of the specific pore.

Porosity of the edible base material is not particularly limited, but is, for example, 50% or more, preferably 70% or more, more preferably 80% or more. Upper limit for the porosity of the edible base material is not particularly limited, but is, for example, 99%. The porosity of the edible base material can be calculated, for example, by obtaining volume and weight of the edible base material to be evaluated and substituting the obtained volume and weight into the following formula (1). In the formula (1), V means volume (cm³), W means weight (g), and D means true density of the edible base material (g/cm³). The true density can be calculated, for example, from volume and weight of a solid obtained by performing steps (I) to (iv) without whisking (without foaming) solution (S) in the abovementioned production method. The true density can also be calculated based on gravity of each component contained in the edible base material.

Porosity (%)=100×[V−(W/D)]/V (1)

In the present invention, shape of the edible base material is not particularly limited, but can be appropriately adjusted according to shape of a cultured meat to be produced. As an example, the edible base material may be a sheet shaped or cube shaped with a thickness of 1 to 30 mm or irregularly shaped.

Apparent density of the edible base material is not particularly limited, but is, for example, 0.8 g/cm³ or less, preferably 0.5 g/cm³ or less, more preferably 0.3 g/cm³ or less, and further more preferably 0.1 g/cm³ or less. Lower limit of the apparent density is not particularly limited, but is, for example, 0.01 g/cm³. The apparent density of the base material can be calculated from volume and weight of the base material to be evaluated.

In the present invention, “adhesion improver” refers to an agent to improve adhesion of cells and base material. In the present invention, the adhesion improver may, for example, be laminated onto the base material or may be coated on the base material. In addition, the adhesion improver may be mixed with the component comprising the base material during production of the base material to be integrated with the base material.

One aspect of the present invention relates to a cell culturing scaffolding material containing the edible base material for cell culturing and the adhesion improver containing the edible plant-derived component of the present invention. In the present invention, the method of applying the adhesion improver in the cell culturing scaffolding material is not particularly limited, but a layer containing the adhesion improver may be laminated on the surface of the edible base material, or the adhesion improver may be kneaded into the edible base material. In a embodiment of the present invention, the cell culturing scaffolding material of the present invention has a layer that said adhesion improver is laminated on the surface of the edible base material.

In addition, if the layer containing adhesion improver is laminated, a strong film may be formed by gelling during drying, or a fragile film may be formed without gelling.

In the present invention, a thickness of the layer containing said adhesion improver is not particularly limited, but is, for example, 1 nm or more. The thickness of the layer containing said adhesion improver is not particularly limited, but is, for example, 500 μm or less, 100 μm or less, 10 μm or less, 5 μm or less, 3 μm or less, 1 μm or less, 500 nm or less, 300 nm or less. If the thickness of the layer containing said adhesion improver exceeds 500 μm, texture is affected.

Weight of the adhesion improver relative to that of the edible base material is not particularly limited, but is 0.01% to 50%, preferably 0.1% to 30%, more preferably 1% to 10%. In addition, the adhesion improver only needs to contribute to cell adhesion to the cell culturing base material, and the adhesion improver may remain on the edible base material while the cells are cultured on the cell culturing scaffolding material, or part or all may outflow or elute into culture medium.

One aspect of the present invention relates to a tissue body in which cells are cultured on the cell culturing scaffolding material of the present disclosure. In the present invention, cells to be cultured on the cell culturing scaffolding material are not particularly limited, but are, for example, stem cells, progenitor cells, stromal cells, muscle progenitor cells, fibroblasts, pericytes, endothelial cells (such as aortic endothelial cells and skeletal microvascular endothelial cells), smooth muscle cells, fat cells, etc., preferably smooth muscle cells, fibroblasts, fat cells and stem cells, most preferably smooth muscle cells and fibroblasts.

In the present invention, the term “stem cells” as used herein includes mesenchymal stem cells (MSCs), embryonic stem cells (ESCs), adult stem cells, differentiated ESCs, differentiated adult stem cells, and induced pluripotent stem cells (iPSCs). The term “progenitor cells” as used herein refers to cells that can produce differentiated cells in multiple lineages, such as myoblasts, fibroblasts, fat cells, stromal cells, fibroblasts, and pericytes, smooth muscle cells, and endothelial cells. “Progenitor cells” typically differ from stem cells in that they do not have extensive self-renewal capabilities.

In one embodiment of the present invention, cells are derived from stem cells such as pluripotent embryonic stem cells. In one embodiment of the present invention, cells are mesenchymal stem cells (MSCs). As is known in the art, MSCs can be muscle cells, fat cells, osteocytes, and chondrocytes. In another embodiment of the present invention, cells are induced pluripotent stem cells (iPSCs). In yet another embodiment of the present invention, the cells are derived from totipotent embryonic stem cells such as cells from the blastocyst stage, fertilized egg, placenta, or umbilical cord of animals.

In one embodiment of the present invention, the cells are progenitor cells. In one embodiment of the present invention, the cells are progenitor cells of myoblasts or those of fat cells.

In one embodiment of the present invention, progenitor cells are cultured in monoculture. In one embodiment of the present invention, progenitor cells are differentiated in monoculture. In one embodiment of the present invention, progenitor cells are differentiated in monoculture and then seeded on a cell culturing scaffolding material that is incubated with multiple cells according to the method of the present invention. In one embodiment of the present invention, mesenchymal stem cells are cultured and differentiated to myoblasts, and then the differentiated myoblasts are seeded onto the cell culturing scaffolding material and subsequently incubated. Methods for culturing progenitor cells and inducing differentiation into mature cells are known in the art.

In one embodiment of the present invention, cells are obtained from living animals and cultured as primary cell lines. In other embodiment of the present invention, cells may be obtained by biopsy or may be cultured ex vivo. In yet another embodiment of the present invention, cells are obtained from commercial sources.

In one embodiment of the present invention, the cell culturing scaffolding material of the present invention is a scaffold for culturing muscle progenitor cells in vitro or ex vivo, and for differentiating muscle progenitor cells into specific type of muscle cells such as skeletal muscle cells or smooth muscle cells. In addition, the cell culturing scaffolding material of the present invention may be used to culture fibroblasts, which can secrete extracellular molecules that form an extracellular matrix (ECM) that provides additional structural and mechanical support to the cells.

In one embodiment of the present invention, the cell culturing scaffolding material of the present invention may be used for culturing fat cells, which can provide a specific flavor and texture.

In one embodiment of the present invention, the cell culturing scaffolding material may be used to culture endothelial cells such as aortic endothelial cells and skeletal microvascular endothelial cells, or capillary endothelial formed by the endothelial cells.

In the present invention, the cell culturing scaffolding material may be sterilized prior to seeding or incubating cells. In the present invention, the sterilization is not particularly limited, but is performed, for example, by ethanol or by gamma irradiation.

In the present invention, cell seeding and/or culturing is performed in the presence of cell culturing medium, which may contain growth factor, cytokine, bioactive agent, nutrient, amino acid, antibiotic compound, anti-inflammatory compound, or any combination thereof.

In one embodiment of the present invention, the cell culturing scaffolding material of the present invention may contain a composition that is suitable for cell growth. The composition that is suitable for cell growth is not particularly limited, but include, for example, growth factor, cytokine, bioactive agent, nutrient, amino acid, antibiotic compound, anti-inflammatory compound, natural dye, and perfume.

The growth factor that can be used in the present invention is not particularly limited, but include, for example, platelet-derived growth factor (PDGF) and insulin-like growth factor (IGF-1). The growth factor is not particularly limited, but includes, PDGF (e.g., PDGF AA, PDGF BB); IGF (e.g., IGF-I, IGF-II); fibroblast growth factor (FGF) (e.g., acidic FGF, basic FGF, beta-endothelial cell growth factor, FGF 4, FGF 5, FGF 6, FGF 7, FGF 8, and FGF 9); transforming growth factor (TGF) (e.g., TGF-P1, TGFβ1.2, TGF-β2, TGF-β3, TGF-β5); bone morphogenetic protein (BMP) (e.g., BMP 1, BMP 2, BMP 3, BMP 4); vascular endothelial growth factor (VEGF) (e.g., VEGF, placental growth factor); epidermal growth factor (EGF) (e.g., EGF, amphiregulin, betacellulin, heparin-binding EGF); interleukin (e.g., IL-1, IL-2, IL-3, IL-4, IL-5, IL-6, IL-7, IL-8, IL-9, IL-10, IL-11, IL-12, IL-13, IL-14); colony stimulating factor (CSF) (e.g., CSF-G, CSF-GM, CSF-M); nerve growth factor (NGF); stem cell factor; hepatocyte growth factor, and ciliary neurotrophic factor, etc.

In the present invention, the cells cultured on the cell culturing scaffolding material are not particularly limited, but are cells derived from mammalian animals, avian animals, fish animals, reptilian animals, amphibian animals, and invertebrate animals (such as crustaceans). In the present invention, cells of mammalian animals include cells of cattle, pigs, sheep, horses, bears, goats, rabbits, antelopes, bison, boars, whales, dolphins, beavers, sea otters, dugongs, manatees, seals, sea lions, walruses, weasels, camels, reindeer, deer, elephants, elks, foxes, giraffes, ibex, kangaroos, lions, llamas, moose, peccaries, squirrels, tigers, yaks, zebras, etc.

In the present invention, cells of avian animal include cells of chickens, ducks, turkeys, emus, geese, grouse, ostriches, pheasants, pigeons, quail, etc.

In the present invention, cells of fish animal include cells of tunas, sharks, rays, anglerfishes, sunfishes, marlins, mackerels, horse mackerels, bonitos, sea basses, mackerels, red sea breams, flatfishes, flounders, eels, herrings, salmons, killer whales, sardines, red snappers, blow fishes, basses, catfishes, carps, cods, groupers, haddocks, halibuts, herrings, mahi, swordfishes, orange roughies, perches, pikes, Alaska pollocks, sardines, snappers, swordfishes, tilapias, trouts, walleyes, etc.

In the present invention, cells of reptilian animal include cells of snakes, alligators, turtles, etc. In the present invention, cells of amphibian animal include cells of frogs, etc.

In the present invention, cells of crustacean animal include cells of shrimps, crabs, hills, hermit crabs, crayfishes, lobsters, etc

In the present invention, cells of other invertebrate animal include cells of clams, abalones, seahares, oysters, turban shells, freshwater clams, hard clams, scallops, soletellina diphos, mussels, sea urchins, ascidians, etc.

In the present invention, cells are seeded at a cell density that is favorable for their growth. In one embodiment of the present invention, cells are seeded simultaneously or consecutively. In one embodiment of the present invention, cells are seeded at a cell density of 10³ to 10⁷ cells/cm². In one embodiment of the present invention, a cell density when seeded and the one to be incubated are approximately the same.

In one embodiment of the present invention, coverage of cells on the surface of the cell culturing scaffold is at least 5%, at least 20%, at least 35%, at least 50%, at least 70%, at least 85%, at least 90%, at least 95%, or at least 99%.

In one embodiment of the present invention, cells are seeded so that the coverage of cells on the surface of the cell culturing scaffold to be 1 to 10%, 5 to 20%, 15 to 35%, 30 to 50%, 40 to 65%, 60 to 85%, 80 to 90%, 90 to 100%, or any range between thereof.

In one embodiment of the present invention, the tissue body is for food. In one embodiment of the present invention, the tissue body of the present invention is intended for consumption by human, non-human animal, or both of them, preferably by human. In other embodiment of the present invention, the tissue body of the present invention is intended for consumption by non-human animal, and is used as, for example, animal feed such as livestock feed, aquaculture feed, or household pet feed.

In the following, the present invention will be described in more detail with examples, but the present invention is not limited thereby.

### EXAMPLES

### 1. Preparation of Base Material

Needling was performed on alginate wound dressing (Sorbsan Flat No. 1, 50 mm×50 mm (made by ALCARE)) using a felt puncher (made by Crobar or Fujikyu) set with two needles, to enhance fiber entwinement. The needling was performed on a 28.5 mm square alginate wound dressing by inserting and removing two needles several times per site, at a pitch of 1.5 mm, for a total of 400 sites. The alginate wound dressing on which needling was performed was immersed in a sodium alginate solution adjusted to 0.1%. The alginate wound dressing was then removed and thoroughly wiped dry. It was then immersed in a 100 mM calcium chloride solution for 5 minutes to gelate alginate. The alginate wound dressing was then removed, thoroughly wiped dry, and dried at 90° C. for 1 hour. This was used as the base material for tests.

### 2. Preparation and Coating of an Adhesion Improver

Aqueous solutions or aqueous dispersions containing various edible plant-derived materials at the concentrations listed in Tables 1 to 3 were prepared. The base material prepared in example 1 was immersed in the prepared aqueous solution or aqueous dispersion for 5 minutes. The base material was then removed, thoroughly wiped dry, and dried at 90° C. for 1 hour.

Isolated soy protein A to D used in the experiment are protein isolated from defatted soy milk, and contains, by weight percent, 58.9% to 91.5% of protein, 2.2% to 31.3% of carbohydrate, and 0.2% to 31% of fat. In addition, the isolated soy protein A to C have different gel strengths, and assuming that gel strength of the isolated soy protein A is 1, gel strength of the isolated soy protein B is about 0.5, and that of the isolated soy protein C is about 0.3. In addition, the isolated soy protein C contains about 10% of peptide with a molecular weight of 10,000 or less. In the experiment, New Fujipro SEH (FUJI OIL) was used as the isolated soy protein A, Fujipro-F (FUJI OIL) as the isolated soy protein B, New Fujipro-KM (FUJI OIL) as the isolated soy protein C, and soy protein (DAIICHI-KASEI) as the isolated soy protein D.

Prepared soybean milk powder used in the experiment is dried defatted soybean milk and contains, by weight percent, 2.1% of protein, 27.1 to 28.3% of carbohydrate, and 24.6 to 24.9% of fat. Soyafit (FUJI OIL) was used in the experiment.

Garden pea protein used in the experiment is a condensed and processed protein component in which fiber and starch are removed from seed of yellow pea plant, and contains, by weight percent, 84% of protein, 3% of starch carbohydrate, 1% of fat, and 15% of ash and mineral. Protein GP (DAIICHI-KASEI) was used in the experiment. Extract of seed of Vitis vinifera contains 90% of protoanthocyanidin, and Vinopherone (TOKIWA PHYTOCHEMICAL CO., LTD.) was used in the experiment. Wheat protein is protein extracted from wheat and contains primarily gliadin and glutenin. Grue Wheat Protein A-glu K (Glico Nutrition) was used in the experiment.

Soy meat used in the experiment is processed from the water-insoluble component of defatted soy milk and contains, by weight percent, 41.4 to 74.8% of protein, 15.5 to 44.5% of carbohydrate, and 0.5 to 2.8% of fat. Apex 950 (FUJI OIL) was used in the experiment.

Ginkgo biloba extract used in the experiment contains 24% of flavone glycoside and 6% of terpene lactone. GINKGOLON-24 (TOKIWA PHYTOCHEMICAL CO., LTD.) was used in the experiment.

Maca extract used in the experiment contains 2.4% of benzyl glucosinolate. Maca extract powder FT (TOKIWA PHYTOCHEMICAL CO., LTD.) was used in the experiment.

Ginger extract used in the experiment contains 1.7% of polyphenol. Gingerwarmer (TOKIWA PHYTOCHEMICAL CO., LTD.) was used in the experiment.

Soybean saponin used in the experiment is a soybean extract, and contains 85% of saponin. Soybean Extract I (TOKIWA PHYTOCHEMICAL CO., LTD.) was used in the experiment.

Broad bean protein used in the experiment contains 90% of protein. Favaprotein fiber (Bio Actives Japan) was used in the experiment.

Salacia extract used in the experiment contains 20% of tannin and 5% of triterpene. Salacia Reticulata Extract (Bio Actives Japan) was used in the experiment.

Ginseng extract used in the experiment contains 15% of ginsenoside. American jinseng extract (TAMA BIOCHEMICAL) was used in the experiment.

Tea extract used in the experiment contains 40% of polyphenol. Green tea extract-40 (TAMA BIOCHEMICAL) was used in the experiment.

Corn protein used in the experiment contains 85% of protein. Kobayashi Zein DP-N (KOBAYASHI PERFUMERY) was used in the experiment.

Korean ginseng extract used in the experiment contains 80% of ginsenoside. Korean ginseng extract (80%) (SANCT) was used in the experiment.

Olive extract used in the experiment contains 10% of polyphenol. Olive fruit extract HT-6 (Mitsubishi Chemical) was used in the experiment.

Tannase used in the experiment contains 100% of tannase. Tannase (Mitsubishi Chemical) was used in the experiment.

Psyllium seed gum used in the experiment was purified psyllium seed gum made by the alcohol precipitation method, and SOALLIUM PG200 (Mitsubishi Chemical) was used in the experiment.

Powder Shiitake Extract HD-1 (Sato Foods Industries) was used as shiitake extract in the experiment.

Tamogi mushroom extract used in the experiment is hot air-dried tamogi mushroom hard tip, and powdered Bio-god powder (Three B) was used in the experiment.

Stedygro Soy SESOM (Oriental Yeast) was used as soy peptone in the experiment.

YEAST EXTRACT ULTRAFILTERED POWDER (Oriental Yeast) was used as yeast extract in the experiment.

YEAST PEPTONE HYP-A 581 (Oriental Yeast) was used as yeast peptone in the experiment.

Base material coated with various edible plant-derived materials was stamped out to make test specimen of 6 mm diameter disk.

### 3. Culture Test

(1) Culture Test Using Mouse Cells

Specimens from Examples 1 to 29 were sterilized by immersing in a 70 w/w % ethanol solution and allowing them to stand for 30 minutes. Ethanol was then removed by washing them three times with ultrapure water. The specimens were placed in a 96-well plate (Nunc (trademark) MicroWell (trademark) 96-Well #167008) and each 100 μL of culture medium (DMEM (High glucose) D6546 (Sigma)) in which final concentration of 4 mM L-Glu (L-Glu 25030081 (Thermo)), 10% FBS (PBS 10270 (Thermo)), and 100 units/mL penicillin streptomycin (#168-23191 (Wako)) was respectively supplemented were added, then the plate was allowed to stand for 15 minutes. Culture medium that was not soaked were then removed. Then, a cell solution containing NIH3T3 cells, mouse fetus skin cells, at a density of 1×10⁵ to 1×10⁷ cells/cm 2 was slowly dropped and allowed to stand for 30 minutes. Then, sufficient amount of culture medium was supplemented to soak the base material, and culturing was started at 5% CO² and 37° C. One day later, the base materials were moved to a new plate, and the culture medium were replaced every 3 days for a total of 7 days of continued culture at 5% CO² and 37° C.

Similar culture tests were performed for collagen sponge (honeycomb type CSH-96 (KOKEN)) (referred to Reference Example 1), the base material prepared in example 1. (referred to Comparative Example 1), and two types of soy meat A and B (referred to Comparative Examples 2 and 3), each of which are without coating of edible plant-derived component.

Collagen Sponge is Processed and Lyophilized Atelocollagen.

For soy meat A, VegePlus 2900 (made by FUJI OIL) was used, which has 7.0% of moisture, 51.7% of protein, 0.8% of fat, 33.0% of carbohydrate, and 7.5% of ash, and is relatively soft in firmness. For soy meat B, Apex 950 (made by FUJI OIL) was used, which has 7.0% of moisture, 63.0% of protein, 1.2% of fat, 23.0% of carbohydrate, and 5.8% of ash, and is standard in firmness.

(2) Culture Test Using Coho Salmon Cells

Specimens from Examples 30 to 46 were sterilized by immersing in a 70 w/w % ethanol solution and allowing them to stand for 30 minutes. Ethanol was then removed by washing them three times with ultrapure water. These specimens were placed in a 96-well plate (Nunc (trademark) MicroWell (trademark) 96-Well #167008) and each 100 μL of culture medium (Minimum Essential Medium Eagle (MEME) (M4655-500ML) (Sigma)) in which final concentration of 2 mM L-Glu (L-Glu 25030081 (Thermo)), 10% FBS (FBS 10270 (Thermo)), 1% Non Essential Amino Acid (M7145-100ML (Sigma)), and Penicillin-Streptomycin solution (×1) (#168-23191 (Wako)) was respectively supplemented were added, then the plates was allowed to stand for 15 minutes. Culture medium that was not soaked were then removed. Then, a cell solution containing CSE119 cells, coho salmon cells, at a density of 1×10⁵ to 1×10⁷ cells/cm² was slowly dropped and allowed to stand for 30 minutes. Then, sufficient amount of culture medium was supplemented to soak the base material, and culturing was started at 5% CO² and 20° C. One day later, the base materials were moved to a new plate, and the culture medium were replaced every 3 days for a total of 14 days of continued culture at 5% CO² and 20° C.

### 4. Cell Number Evaluation by CTG Assay

After culturing, 100 μL of cell number evaluation reagent (CellTiter-Glo (registered trademark) 2.0 Cell Viability Assay G9243 (Promega)) was added to each well and gently stirred for about 2 minutes. 200 μL of supernatant from each well was moved to a 96-well plate for luminescence measurement (Perkin Elmer (trademark) OptiPlate-96 #6005299), luminescence was measured according to standard protocol for luminescence measurement of plate reader (Perkin Elmer EnSight), and cell number was calculated using previously prepared calibration curve. Cell number was measured with automated cell counter Thermo Countess (registered trademark) II FL. Calculated cell numbers were shown as percentage to the cell number calculated in the collagen sponge (honeycomb type CSH-96 (KOKEN)) (Reference Example 1).

The results are shown in Tables 1 to 3.

TABLE 1

| | | | | | Cell |
|---|---|---|---|---|---|
| | Base | Coating | | | number |
| | material | | Product name/ | | (To |
| | Material | Material | Manufacturer | Content | collagen) |
| Example 1 | Alginic non- | Isolated soy | New Fujipro SEH/ | 0.10% | 54% |
| | woven fabric | protein A | FUJI OIL | | |
| Example 2 | Alginic non- | Isolated soy | New Fujipro SEH/ | 1.00% | 47% |
| | woven fabric | protein A | FUJI OIL | | |
| Example 3 | Alginic non- | Isolated soy | New Fujipro SEH/ | 0.01% | 44% |
| | woven fabric | protein A | FUJI OIL | | |
| Example 4 | Alginic non- | Isolated soy | Fujipro F/ | 0.10% | 52% |
| | woven fabric | protein B | FUJI OIL | | |
| Example 5 | Alginic non- | Isolated soy | New Fujipro KM/ | 0.10% | 41% |
| | woven fabric | protein C | FUJI OIL | | |
| Example 6 | Alginic non- | Processed soy | Soyafit/ | 0.10% | 34% |
| | woven fabric | milk powder | FUJI OIL | | |
| Example 7 | Alginic non- | Isolated soy | Soy protein/ | 0.10% | 45% |
| | woven fabric | protein D | DAIICHI-KASEI | | |
| Example 8 | Alginic non- | Garden pea | Protein GP/ | 0.10% | 54% |
| | woven fabric | protein | DAIICHI-KASEI | | |
| Example 9 | Alginic non- | Grape seed | Vinopherone/ | 0.10% | 64% |
| | woven fabric | extract | TOKIWA | | |
| | | | PHYTOCHEMICAL | | |
| Example 10 | Alginic non- | Wheat protein | Grue Wheat Protein | 0.10% | 13% |
| | woven fabric | | A-glu K/ | | |
| | | | Glico Nutrition | | |
| Example 11 | Alginic non- | Insoluble soy | Apex 950/ | 0.10% | 10% |
| | woven fabric | protein | FUJI OIL | | |

| | | | |
|---|---|---|---|
| Comparative | Alginic non- | No coating | 7% |
| Example 1 | woven fabric | | |
| Comparative | Soy meat A | No coating | 3% |
| Example 2 | VegePlus 2900 | | |
| | (FUJI OIL) | | |
| Comparative | Soy meat B | No coating | 8% |
| Example 3 | Apex 950 | | |
| | (FUJI OIL) | | |

TABLE 2

| | Coating | | | |
|---|---|---|---|---|
| | | Product name/ | | Cell number |
| | Material | Manufacturer | Content | (To collagen) |
| Example 12 | Ginkgo biloba | GINKGOLON-24/ | 3.00% | 41% |
| | extract | TOKIWA PHYTOCHEMICAL | | |
| Example 13 | Maca extract | Maca extract powder FT/ | 1.00% | 35% |
| | | TOKIWA PHYTOCHEMICAL | | |
| Example 14 | Ginger extract | Gingerwarmer/ | 3.00% | 45% |
| | | TOKIWA PHYTOCHEMICAL | | |
| Example 15 | Soybean saponin | Soybean Extract I/ | 1.00% | 41% |
| | | TOKIWA PHYTOCHEMICAL | | |
| Example 16 | Broad bean protein | Favaprotein fiber/ | 1.00% | 47% |
| | | Bio Actives Japan | | |
| Example 17 | Salacia extract | Salacia reticulata extract/ | 0.10% | 33% |
| | | Bio Actives Japan | | |
| Example 18 | Ginseng extract | American jinseng extract/ | 0.10% | 50% |
| | | TAMA BIOCHEMICAL | | |
| Example 19 | Green tea extract | Green tea extract-40/ | 0.10% | 29% |
| | | TAMA BIOCHEMICAL | | |
| Example 20 | Corn protein | Kobayashi Zein DP-N/ | 1.00% | 50% |
| | | KOBAYASHI PERFUMERY | | |
| Example 21 | Korean ginseng | Korean ginseng extract (80%)/ | 0.10% | 19% |
| | extract | SANCT | | |
| Example 22 | Olive extract | Olive fruit extract HT-6/ | 0.10% | 13% |
| | | Mitsubishi Chemical | | |
| Example 23 | Tannase | Tannase/ | 0.10% | 16% |
| | | Mitsubishi Chemical | | |
| Example 24 | Psyllium seed gum | SOALLIUM PG200/ | 0.10% | 25% |
| | | Mitsubishi Chemical | | |
| Example 25 | Shiitake extract | Powder Shiitake Extract HD-1/ | 3.00% | 18% |
| | | Sato Foods Industries | | |
| Example 26 | Tamogi mushroom | Powdered Bio-god powder/ | 3.00% | 40% |
| | extract | Three B | | |
| Example 27 | Soy peptone | Stedygro Soy SE50M/ | 0.10% | 22% |
| | | Oriental Yeast | | |
| Example 28 | Yeast extract | YEAST EXTRACT | 3.00% | 25% |
| | | ULTRAFILTERED POWDER/ | | |
| | | Oriental Yeast | | |
| Example 29 | Yeast peptone | YEAST PEPTONE HYP-A 581/ | 3.00% | 45% |
| | | Oriental Yeast | | |

TABLE 3

| | Coating | | | |
|---|---|---|---|---|
| | | Product name/ | | Cell number |
| | Material | Manufacturer | Content | (To collagen) |
| Example 30 | Ginkgo biloba | GINKGOLON-24/ | 1.00% | 35% |
| | extract | TOKIWA PHYTOCHEMICAL | | |
| Example 31 | Maca extract | Maca extract powder FT/ | 3.00% | 44% |
| | | TOKIWA PHYTOCHEMICAL | | |
| Example 32 | Ginger extract | Gingerwarmer/ | 3.00% | 49% |
| | | TOKIWA PHYTOCHEMICAL | | |
| Example 33 | Soybean saponin | Soybean Extract I/ | 1.00% | 40% |
| | | TOKIWA PHYTOCHEMICAL | | |
| Example 34 | Broad bean protein | Favaprotein fiber/ | 3.00% | 53% |
| | | Bio Actives Japan | | |
| Example 35 | Salacia extract | Salacia reticulata extract/ | 0.10% | 54% |
| | | Bio Actives Japan | | |
| Example 36 | Ginseng extract | American jinseng extract/ | 0.10% | 22% |
| | | TAMA BIOCHEMICAL | | |
| Example 37 | Green tea extract | Green tea extract-40/ | 0.10% | 35% |
| | | TAMA BIOCHEMICAL | | |
| Example 38 | Corn protein | Kobayashi Zein DP-N/ | 1.00% | 19% |
| | | KOBAYASHI PERFUMERY | | |
| Example 39 | Korean ginseng | Korean ginseng extract (80%)/ | 1.00% | 63% |
| | extract | SANCT | | |
| Example 40 | Olive extract | Olive fruit extract HT-6/ | 0.10% | 56% |
| | | Mitsubishi Chemical | | |
| Example 41 | Tannase | Tannase/ | 1.00% | 60% |
| | | Mitsubishi Chemical | | |
| Example 42 | Psyllium seed gum | SOALLIUM PG200/ | 0.10% | 54% |
| | | Mitsubishi Chemical | | |
| Example 43 | Shiitake extract | Powder Shiitake Extract HD-1/ | 0.10% | 27% |
| | | Sato Foods Industries | | |
| Example 44 | Tamogi mushroom | Powdered Bio-god powder/ | 1.00% | 58% |
| | extract | Three B | | |
| Example 45 | Soy peptone | Stedygro Soy SE50M/ | 0.10% | 20% |
| | | Oriental Yeast | | |
| Example 46 | Yeast extract | YEAST EXTRACT | 0.10% | 15% |
| | | ULTRAFILTERED POWDER/ | | |
| | | Oriental Yeast | | |

A significant increase in the number of cells cultured was observed when cells were cultured on a scaffold coated with edible plant-derived component on the base material (Examples 1 to 46) compared to when cells were cultured on the base material without coating of edible plant-derived component (Comparative Example 1) and when cells were cultured on soy meat itself as the scaffold (Comparative Examples 2 and 3).

5. Morphological Observation with Confocal Microscope

After 7 days of culturing, cells were stained with live cell staining reagent (Calcein AM 1 mg/mL in DMSO #349-07201 (Wako)) and nuclear staining reagent (Hoechst 33342 10 mg/mL in H₂O #H3570 (Thermo)) and morphology was observed with confocal microscope (Nikon A1). In two-dimensional image, transmitted light image, Calcein AM stained image (green), and Hoechst stained image (blue) were superimposed, and in three-dimensional image, autofluorescence image (red) was superimposed instead of the transmitted light image.

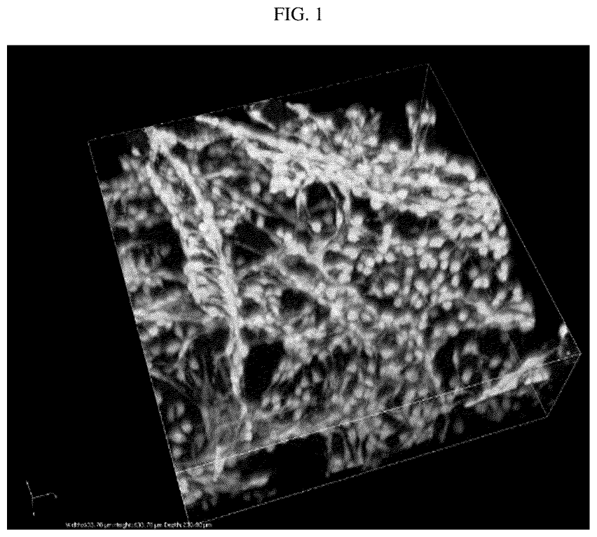

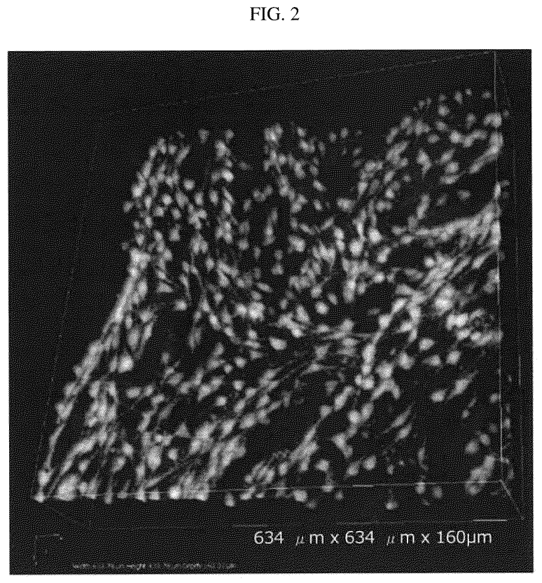

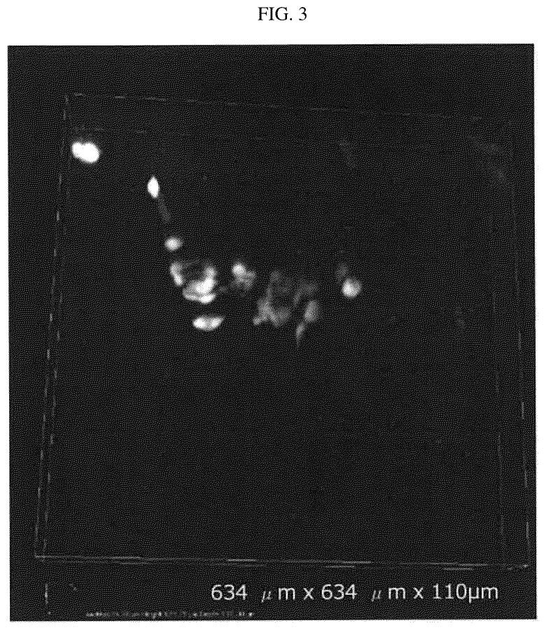

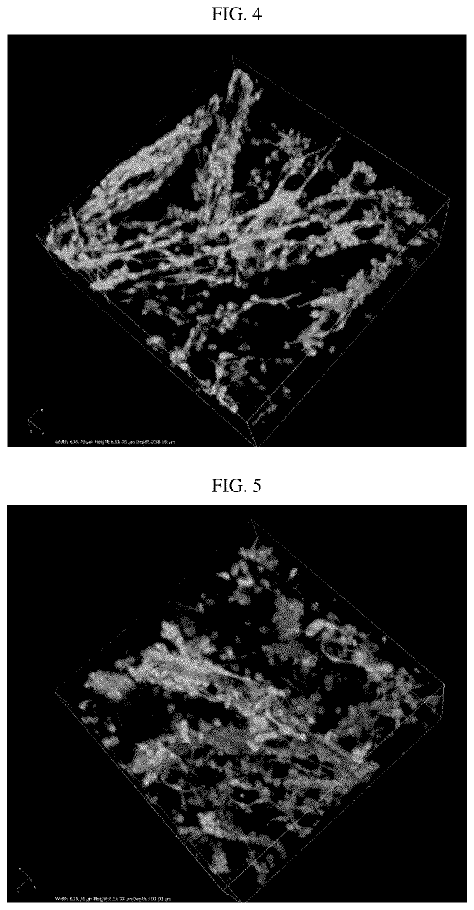

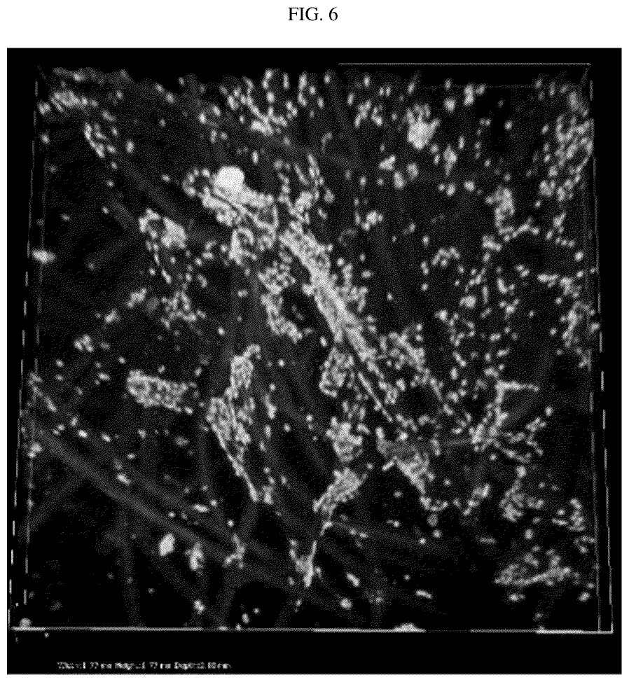

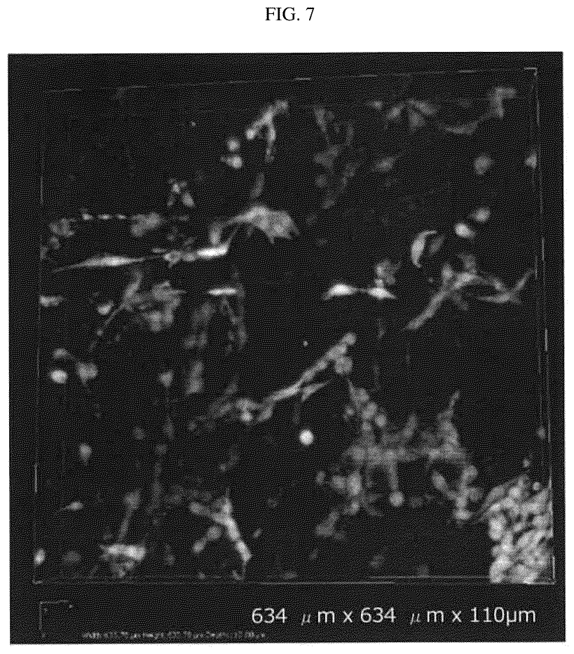

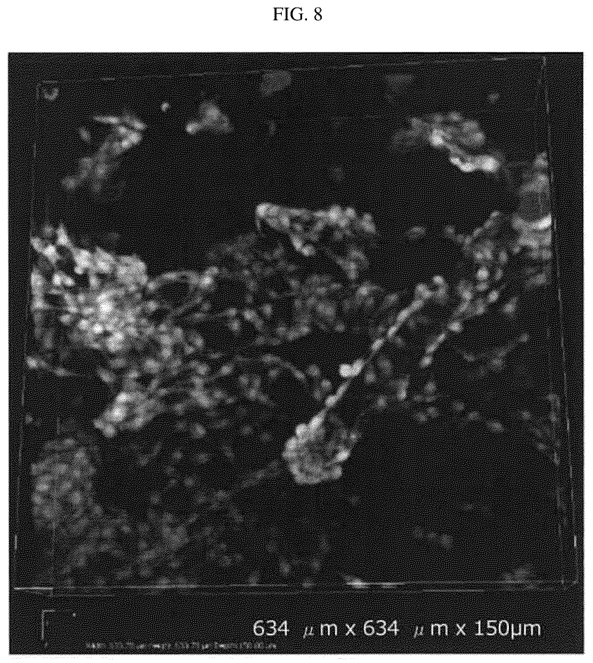

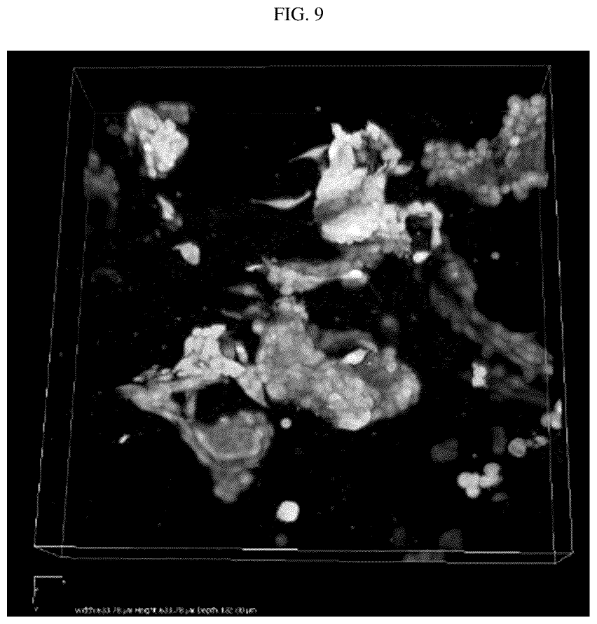

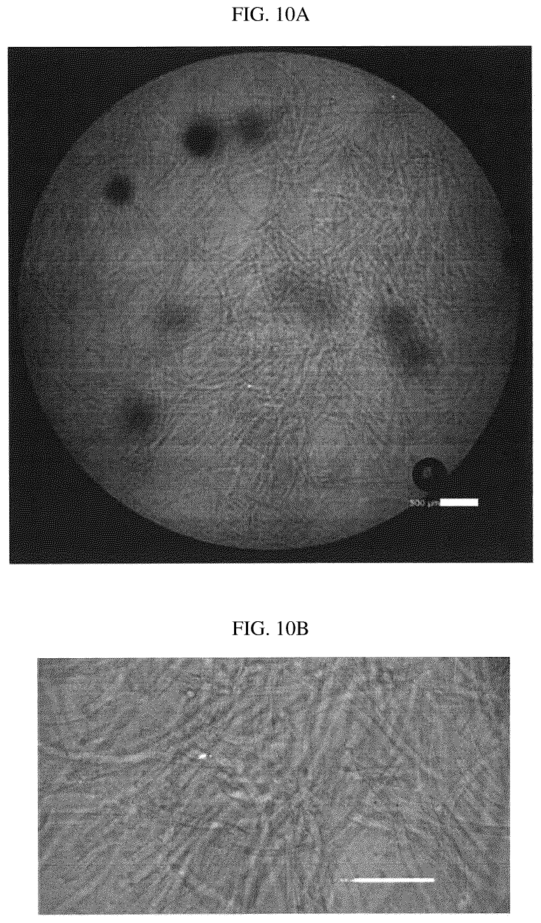

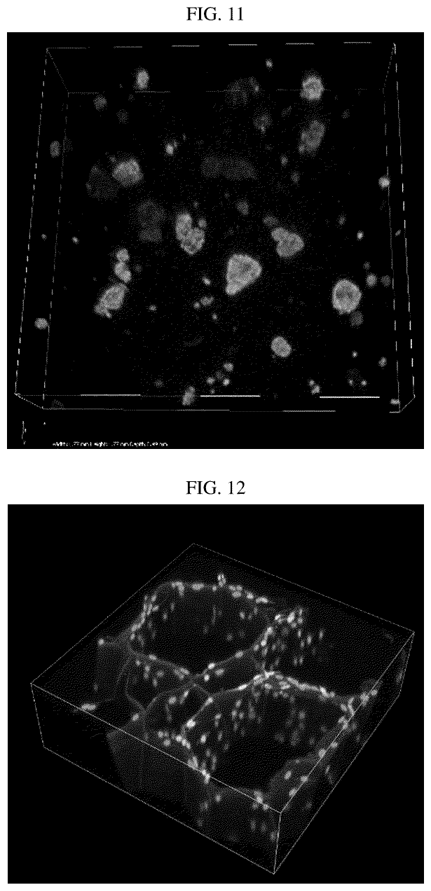

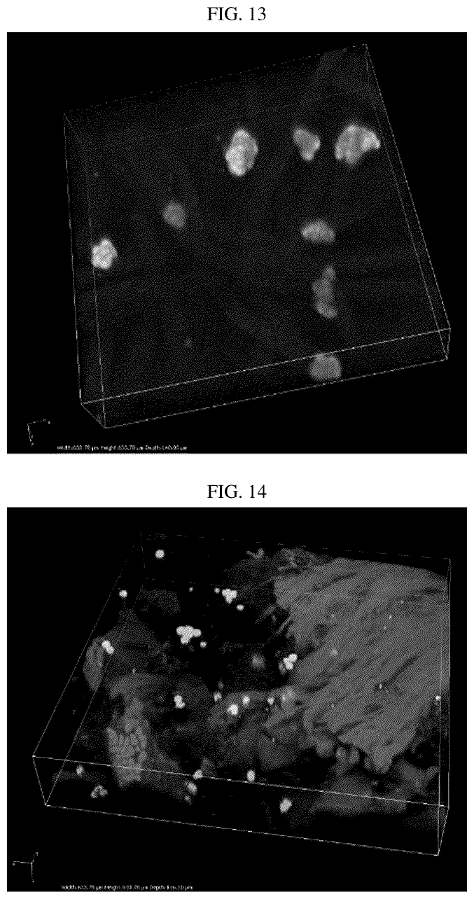

Three-dimensional images of Examples 1 to 9 are shown in FIGS. 1 to 9, two-dimensional images of Example 10 in FIG. 10, a three-dimensional image of Example 11 in FIG. 11, a three-dimensional image of Reference Example 1 in FIG. 12, and three-dimensional images of Comparative Examples 1 to 3 in FIGS. 13 to 15, respectively.

In Comparative Examples 1 to 3 (FIGS. 13 to 15), the cells did not adhere to the base material, and the cells gathered together to form cell aggregation. Cells cannot grow under this situation.

In Examples 1 to 10 (FIGS. 1 to 10), cells adhered to the base material and cytoplasm extended well, confirming cell growth. In Example 11 (FIG. 11), cells adhered to the base material, but extension of cytoplasm was a little less.

### 6. Coating Thickness Measurement Test

Coating thickness of coating layer of the specimen in Example 1 was measured by TOF-SIMS, and coating thickness of coating layer of the specimens in Examples 1 and 2 were measured by SEM.

TRIFT-V (ULVAC-PHI) was used for TOF-SIMS. The specimens were cut into 1 cm square, fixed onto a sample table, irradiated with Bi₃²⁺ as primary ion (primary ion acceleration voltage: 30 kV), and the coating thickness was measured over a measurement area of 30 μm square and 500 μm square. A neutralizing gun for charge correction was used for the measurement.

Phenom prox (Thermo Fisher scientific) was used for SEM. The specimen was cut to show cross section, the cut specimen was fixed onto a sample table, and Au—Pd sputtering processing was performed. The sample table was set in the imaging section and the fiber cross section was observed at a magnification of 6800 times (acceleration voltage 10 kV, vacuum mode). MSP-1S (Vacuum Device Inc.) was used for sputtering device.

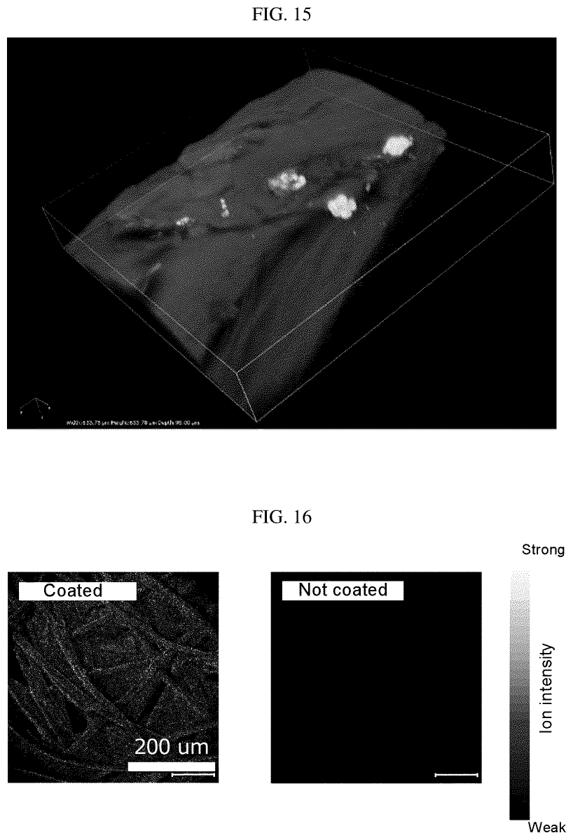

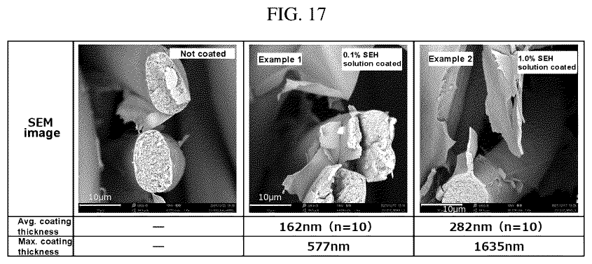

The results are shown in Tables 4. In addition, an image, obtained by TOF-SIMS, mapping C₄H₈N⁺ (m/z70) derived from soy protein (SEH) amino acids is shown in FIG. 16, and an image obtained by SEM (image magnification of 6800 times) is shown in FIG. 17.

TABLE 4

| | | | | Coating thickness (nm) | | |
|---|---|---|---|---|---|---|
| | | | | | SEM | SEM |
| | Coating | | | | (Average | (Max. |
| | | Product name/ | Content | TOF- | coating | coating |
| | Material | Manufacturer | (wt %) | SIMS | thickness) | thickness) |
| Example 1 | isolated soy | New Fujipro | 0.10% | 1 or more | 162 | 577 |
| | protein A | SEH/FUJI OIL | | | | |
| Example 2 | isolated soy | New Fujipro | 1.00% | — | 282 | 1635 |
| | protein A | SEH/FUJI OIL | | | | |

1. An adhesion improver containing edible plant-derived component, that is used in an edible base material for cell culturing.

2. The adhesion improver according to claim 1, wherein the edible plant-derived component is seed-derived.

3. The adhesion improver according to claim 2, wherein the seed is a Fabaceae plant seed.

4. The adhesion improver according to claim 3, wherein the seed is a soybean.

5. The adhesion improver according to claim 1, wherein the edible plant-derived component is isolated soy protein.

6. The adhesion improver according to claim 1, wherein the edible base material contains alginic acid or alginate, glucomannan, or cellulose derivative.

7. The adhesion improver according to claim 1, wherein the edible base material is a porous material.

8. A cell culturing scaffolding material containing the edible base material for cell culturing and the adhesion improver according to claim 1.

9. The cell culturing scaffolding material according to claim 8, wherein a layer containing said adhesion improver is laminated on a surface of the edible base material.

10. The cell culturing scaffolding material according to claim 9, wherein a thickness of the layer containing said adhesion improver is 1 nm or more.

11. A tissue body in which cells are cultured on the cell culturing scaffolding material according to claim 8.

12. The tissue body according to claim 11, wherein the cell is derived from mammal, fish, or crustacean.

13. The tissue body according to claim 11, wherein the tissue body is for food.
